(19) United States
(12) Patent Application Publication
(10) Pub. No.: US 2004/0147613 A1
(43) Pub. Date: Jul. 29, 2004

(54) Use of neuroactive substances for the treatment of parkinsons disease and pharmaceutical combination

(76) Inventor: Ralph Dawirs, Enger (DE)

Correspondence Address: Eric M Dobrusin, Dobrusin & Thennisch, Suite 311, 401 South Old Woodward Avenue, Birmingham, MI, 48009, US

(21) Appl. No.: 10/469,833

(22) PCT Filed: Mar. 8, 2002
(86) PCT No.: PCT/EP02/02571

(30) Foreign Application Priority Data
Mar. 9, 2001 (DE) 101 11 486.9

(51) Int. Cl. A61K 31/137

(52) U.S. Cl. 514/649

(57) ABSTRACT

The invention concerns a use of a first substance which at least partially blocks postsynaptic receptors specific for a given neurotransmitter, and a second substance which initiates or promotes deafferentation of the innervation to be attributed to the neurotransmitter for the treatment of Parkinson's disease or for the production of a drug or a drug combination for the treatment of Parkinson's disease.

The invention further concerns a drug combination.

The invention concerns a use of neuroactive substances for the treatment of Parkinson's disease and for the production of a drug or a drug combination for the treatment of Parkinson's disease. The invention further concerns a drug combination.

The Parkinson syndrome is one of the most frequent neurological illnesses of advanced years. In the group of over 60s about 1%, in particular of men, suffer from the Parkinson syndrome. The Parkinson syndrome manifests itself in numerous symptoms which can be roughly divided into three groups:

1. motor disturbances, for example rigor and tremor;

2. vegetative disturbances, for example increased salivation and tear production, and reduced blood pressure; and

3. psychological disturbances, for example depressive moods and slower thought processes.

Even if to date there are no conclusive concepts in regard to the etiology of that idiopathic illness, it is nonetheless indisputable that a progressive destruction of dopaminergic neurons of the substantia nigra is an essential basic condition for the later occurrence of the symptoms. Within what is to referred to as the ‘motor circuit’ the reduction in nigrostriatal dopamine projection leads to an overweighting of the indirect path by way of the nucleus subthalamicus with the result of excessive inhibition of thalamo-cortical projections.

There are numerous concepts for treatment of the symptoms of Parkinson's disease. That involves using a whole range of medications. Alone or in combination they serve for the functional substitution of the lost dopamine cells in the midbrain; these include: L-dopa, dopamine agonists, MAO-B-inhibitors, COMT-inhibitors, anticholinergics and NMDA-antagonists.

In addition the following neurosurgical treatment processes are used: pallidotomy, thalamotomy, deep brain stimulation and transplantation of dopamine-producing cells. Those processes are invasive to a high degree by virtue of their very nature and entail high risk levels. Even those interventions ultimately do not result in a cure and they do not result in checking the progressive advance of the course of the disease. They can serve for medication relief for a limited time.

What is common to all strategies in the state of the art is that they are directed to treatment of the symptoms of Parkinson's disease. To date no measures are available for preventing and curing Parkinson's disease.

The object of the invention is to eliminate the disadvantages of the state of the art and in particular to provide a use of one or more substances which serve for curing or preventing Parkinson's disease.

That object is attained by the features of the independent claim.

Advantageous configurations and developments of the invention are recited in the appendant claims.

The invention lies in a use of a first substance which at least partially blocks postsynaptic receptors specific for a given neurotransmitter, and a second substance which initiates or promotes deafferentation of the innervation which is to be attributed to the neurotransmitter for treatment of Parkinson's disease or for the production of a drug or a drug combination for treatment of Parkinson's disease. The first appearance of the symptoms of Parkinson's disease is preceded by a very long pre-clinical progressive course. In that pre-clinical phase of unrecognised progressive loss of nigro-striatal projections the dopaminergic system affords an astonishing functional compensation effect. It is only after about 80-90% of the dopaminergic neurons of the substantia nigra are destroyed that this compensation capability is exhausted and the typical symptoms come to light. The use according to the invention exhibits its particular advantages in particular prior to the appearance of the typical symptoms. Blocking of the postsynapses causes in particular preparatory compensatory stimulation of the presynapses. Then deafferentation can serve as ‘initial triggering’ for a subsequent rise in innervation density. The use according to the invention is suitable for inducing reactive homotypical sprouting of dopaminergic fibers in the striatum, with the consequence of age-specifically increased innervation density of dopaminergic fibers in the striatum.

Preferably a substance for blocking postsynaptic dopamine receptors is used as the first substance. That affords compensatory stimulus of the activity of the dopaminergic presynapses so that this affords the prerequisites for deafferentation of innervation which is to be attributed to the neurotransmitter.

In that respect it is particularly advantageous if a dopamine antagonist is used as the first substance. Specific psychopharmaceuticals of that kind are particularly well suited to implementing a blockade in respect of postsynaptic dopamine receptors.

It may also be advantageous to use an antipsychotic as the first substance. The effect of antipsychotics can also directly influence the function of the postsynapses.

It is particularly preferable if haloperidol is used as the first substance. Haloperidol is a specific dopamine antagonist with a particular action on D₂-receptors, which is well known in regard to its basic pharmacological properties. Thus, with suitable adjustment of the dosage, it is possible by means of haloperidol to specifically set a targeted blocking action in respect of the dopaminergic postsynapses and consequently a compensatory stimulus in respect of the activity of the dopaminergic presynapses.

Preferably the second substance used is a substance for increasing the oxidative stresses in nerve cells, in particular in dopamine cells. On the basis of the compensatory stimulus in respect of the activity of the dopaminergic presynapses, which is a consequence of supplying the first substance, the option is afforded of initiating partial deafferentation of dopaminergic innervation, by virtue of the increase of oxidative stress in dopamine cells. That can then induce reactive homotypical sprouting of dopaminergic fibers in the striatum.

It is preferable for an amphetamine derivative to be used as the second substance. Amphetamine derivatives are well researched in terms of their general action on the central nervous system so that this fundamental knowledge can advantageously be put to use in regard to suitably implementing the treatment.

It is particularly preferred if methamphetamine is used as the second substance. With that common pharmacological substance, it is possible by means of sub-threshold dosage to specifically initiate the advantageous slight deafferentation of dopaminergic innervation.

It is particularly useful if the first substance is used prior to the second substance. That therefore initially involves blocking of the postsynaptic dopamine receptors and consequently compensatory stimulus in respect of the activity of the dopaminergic presynapses. That therefore affords the prerequisites for the second substance to be able to initiate deafferentation of dopaminergic innervation, at a low level of dosage.

Preferably the first substance and/or the second substance are used a plurality of times. Repeated use improves the action of the drug.

Depending on the respective particular conditions and the specific aim in mind, it may be preferable if the first substance and/or the second substance are used orally and/or subcutaneously and/or intravenously and/or intraperitoneally. The manner of use is therefore not limited to a specific way of supplying the substances; depending on the respective purpose involved however specific advantages can arise out of given forms of administration. It is particularly advantageous however if the substances are supplied orally as that mode of supply is well known for example in regard to the substances haloperidol and methamphetamine which are preferably supplied.

The invention further concerns a drug combination for the treatment of Parkinson's disease, comprising a first substance which at least partially blocks postsynaptic receptors specific for a given neurotransmitter and a second substance which initiates or promotes deafferentation of the innervation which is to be attributed to the neurotransmitter. Accordingly per se known substances of that kind are involved in a functional unit by virtue of a specifically targeted use. The drug combination which is specified in such general terms can advantageously be configured in accordance with the uses referred to herein, for example by virtue of the first substance involving haloperidol and the second substance involving methamphetamine.

The invention is based on the surprising realisation that the age-governed loss of dopaminergic nerve fibers in the striatum can be checked by the use of per se known pharmacological substances. Innervation density can rise or the drop in innervation density can be delayed or prevented. The basis of the invention is that local and transsynaptic reorganisation processes of the neuronal circuitry pattern are natural properties of the nervous system, the functional significance of which lies in the normal, non-invasive, physiological interaction of the system with the milieu. Besides hormones in particular the neurotransmitters appear to play an important part in regard to structural processes in the central nervous system. Thus the neurotransmitters selectively influence the behaviour of the axonal growth cone in development and progressive and regressive aspects of structural reorganisation processes. In addition the neurotransmitters are involved in processes which control the physiological and pathological cell death of individual neurones. Ongoing reconstruction of the synaptic spectrum is quite evidently of fundamental significance for achieving functional integrity of the mature brain. Accordingly the nervous system can be viewed as an open dynamic system and thus as inevitably communicative-morphogenetic, that is to say in terms of openness for pulses from the environmental conditions the formation of appropriate structural correlates for adapted behaviour of the organisms appears as a property inherent in the nervous system. While the advantages of such a development strategy are apparent, it does however also involve risks: pathological behaviour patterns must also be understood as a consequential result of adaptive but nonetheless aberrant structural development. In that connection major significance will be attributed in the future to the targeted use of the morphogenetic potential of neuroactive substances (systemic structural pharmacology of the CNS). The present invention makes an important contribution in that respect, in relation to a widespread illness. Particularly during ‘critical periods’ of the early development, neuroactive substances have a substantial influence on neurogenesis, in the same manner as their natural cousins such as neurotransmitters. In addition neuropharmaceuticals function as indirect agonists or antagonists by virtue of a non-adaptive increase or reduction in levels of transmitter activity. Now, in particular transient neuronal interlinking patterns and functional states play an important part in terms of ontogenesis of the brain. Those necessary, consecutively changing, structural and functional balances can be detrimentally influenced by neuropharmaceuticals. The most widely differing effects on neurogenesis and behaviour are the consequence. For example expression of the plastic capacities, typical of adult rats, of central DA-receptors ceases after foetal administrations of β-endorphin. In addition it has been found that foetal administrations of the DA-receptor antagonist haloperidol leads to persistent down-regulation of central DA-receptors. Furthermore it has been seen that the chronic administration of haloperidol induces dynamic reorganisation of local synapse populations, for example in the substantia nigra, in the striatum and in the prefrontal cortex. Although hitherto no definitive information is to be afforded in regard to the transmitter specificity of that pharmacologically induced plasticity, there are nonetheless concrete indications. Thus for example a 4-month chronic treatment with haloperidol causes a dramatic rise in the number of GABAergic axosomatic synapses in the medial prefrontal cortex of a rat. In addition there are indications of transsynaptic sprouting of glutamatergic corticostriatal projections after a 14-day chronic treatment with haloperidol. Antidepressants are obviously also in a position to trigger off specific structural processes. Thus chronic treatment with the noradrenaline-reuptake-blocker desipramine induces axonal sprouting of central noradrenergic and probably also dopaminergic cortical projections.

The subject of the invention is the targeted use of the morphogenetic potential of neuroactive substances for the purpose of selective reorganisation of neuronal structures. Reorganisation processes of that kind are proven by the animal experiment described hereinafter.

The living creature on which the experiments were carried out is a Mongolian gerbil (Meriones unguiculatus). The Mongolian gerbil was aged 90 days. By virtue of having been reared under severely restrictive conditions, the brain of the gerbil has a reduced level of innervation density of dopaminergic neurons in the striatum, although the gerbil still does not exhibit any typical Parkinson symptoms. This therefore involves a condition which is comparable to or corresponds to an early stage in the preclinical progress of Parkinson's disease. The substances are intraperitoneally supplied to the Mongolian gerbil in the dosages set out hereinafter. In that respect the following administration was found to be particularly advantageous in terms of amount and sequence, on three successive days.

Day 1:

09:00 hours 5 mg/kg haloperidol

13:00 hours 5 mg/kg haloperidol

17:00 hours 5 mg/kg haloperidol

Day 2:

09:00 hours 5 mg/kg haloperidol

11:00 hours 1 mg/kg methamphetamine

13:00 hours 5 mg/kg haloperidol

17:00 hours 5 mg/kg haloperidol

Day 3:

09:00 hours 5 mg/kg haloperidol

13:00 hours 5 mg/kg haloperidol

17:00 hours 5 mg/kg haloperidol

The specific embodiment described by way of example is summarised in the following Table.

| | |
|---|---|
| Animal | Mongolian gerbil (Meriones unguiculatus) |
| Beginning of the treatment | At the age of 90 days (young adult) |
| Substances | haloperidol in combination with |
| | methamphetamine |
| Dose | haloperidol (5 mg/kg) |
| | methamphetamine (1 mg/kg) |
| Application | intraperitoneal (i.p.) |
| Regime | Day 1: |
| | 09:00 hours 5 mg/kg haloperidol |
| | 13:00 hours 5 mg/kg haloperidol |
| | 17:00 hours 5 mg/kg haloperidol |
| | Day 2: |
| | 09:00 hours 5 mg/kg haloperidol |
| | 11:00 hours 1 mg/kg methamphetamine |
| | 13:00 hours 5 mg/kg haloperidol |
| | 17:00 hours 5 mg/kg haloperidol |
| | Day 3: |
| | 09:00 hours 5 mg/kg haloperidol |
| | 13:00 hours 5 mg/kg haloperidol |
| | 17:00 hours 5 mg/kg haloperidol |

The comparative results significantly revealed compensation or overcompensation of the age-governed drop in innervation density in the striatum. The dosage in relation to a human being, in the case of haloperidol, is for example in a range of between 0.05 and 1 mg/kg. Methamphetamine is administered for example in a dosage of between 0.01 and 1 mg/kg. It can be particularly useful to match the dosages of haloperidol and methampetamine to each other. The ratio of the dosages of haloperidol to methamphetamine can be in a range of between 1:1 and 10:1, preferably in a range of between 4:1 and 6:1.

To sum up it can be said that neuroactive substances are particularly suitable for intervening in the natural plastic structural processes of the brain.

The features of the invention disclosed in the foregoing description and in the claims can be essential for carrying the invention into effect both individually and also in any combination.

1. Use of
a first substance which at least partially blocks postsynaptic receptors specific for a given neurotransmitter, and
a second substance which initiates or promotes deafferentation of the innervation to be attributed to the neurotransmitter,
for the treatment of Parkinson's disease.

2. Use of
a first substance which at least partially blocks postsynaptic receptors specific for a given neurotransmitter, and
a second substance which initiates or promotes deafferentation of the innervation to be attributed to the neurotransmitter,
for the production of a drug or a drug combination for the treatment of Parkinson's disease.

3. A use as set forth in claim 1 or claim 2 characterised in that a substance for blocking postsynaptic dopamine receptors is used as the first substance.

4. Use as set forth in one of the preceding claims characterised in that a dopamine antagonist is used as the first substance.

5. Use as set forth in one of the preceding claims characterised in that an antipsychotic is used as the first substance.

6. Use as set forth in one of the preceding claims characterised in that haloperidol is used as the first substance.

7. Use as set forth in one of the preceding claims characterised in that a substance for increasing the oxidative stress in nerve cells, in particular in dopamine cells, is used as the second substance.

8. Use as set forth in one of the preceding claims characterised in that amphetamine derivatives are used as the second substance.

9. Use as set forth in one of the preceding claims characterised in that metamphetamine is used as the second substance.

10. Use as set forth in one of the preceding claims characterised in that the first substance is used prior to the second substance.

11. Use as set forth in one of the preceding claims characterised in that the first substance and/or the second substance are used a plurality of times.

12. Use as set forth in one of the preceding claims characterised in that the first substance and/or the second substance are used orally and/or subcutaneously and/or intravenously and/or intraperitoneally.

13. Use as set forth in one of the preceding claims characterised in that the treatment of Parkinson's disease acts preventatively on symptoms of Parkinson's disease.

14. A drug combination comprising
a first substance which at least partially blocks postsynaptic receptors specific for a given neurotransmitter, and
a second substance which initiates or promotes deafferentation of the innervation to be attributed to the neurotransmitter,
for the treatment of Parkinson's disease.

15. A drug combination as set forth in claim 14 characterised in that the first substance is a substance for blocking postsynaptic dopamine receptors.

16. A drug combination as set forth in claim 14 or claim 15 characterised in that the first substance is a dopamine antagonist.

17. A drug combination as set forth in one of claims 14 through 16 characterised in that the first substance is an antipsychotic.

18. A drug combination as set forth in one of claims 14 through 17 characterised in that the first substance is haloperidol.

19. A drug combination as set forth in one of claims 14 through 18 characterised in that the second substance is a substance for increasing the oxidative stress in nerve cells, in particular in dopamine cells.

20. A drug combination as set forth in one of claims 14 through 19 characterised in that the second substance is an amphetamine derivative.

21. A drug combination as set forth in one of claims 14 through 20 characterised in that the second substance is methamphetamine.

22. A drug combination as set forth in one of claims 14 through 21 characterised in that when used as specified the first substance is used prior to the second substance.

23. A drug combination as set forth in one of claims 14 through 22 characterised in that when used as specified the first substance and/or the second substance are used a plurality of times.

24. A drug combination as set forth in one of claims 14 through 23 characterised in that when used as specified the first substance and/or the second substance are used orally and/or subcutaneously and/or intravenously and/or intraperitoneally.

25. A drug combination as set forth in one of claims 14 through 24 characterised in that it can be used preventatively in respect of the symptoms of Parkinson's disease.
